(19) United States
(12) Patent Application Publication
(10) Pub. No.: US 2008/0089843 A1
(43) Pub. Date: Apr. 17, 2008

(54) USE OF BIOMARKERS OF INFLAMMATION AS INDICATORS OF DRUG EFFICACY

(75) Inventors: Siviram Pillarisetti, Norcross, GA (US); Sumera Nikhat Hasham, Secunderabad (IN)

Correspondence Address: Reddy Us Therapeutics, Inc, 3065 Northwoods Circle, Norcross, GA, 30071, US

(73) Assignee: REDDY'S LABORATORIES, INC., Bridgewater, NJ (US)

(21) Appl. No.: 11/753,951

(22) Filed: May 25, 2007

Related U.S. Application Data
(60) Provisional application No. 60/808,396, filed on May 25, 2006.

(51) Int. Cl. G01N 33/53

(52) U.S. Cl. 424/9.2

(57) ABSTRACT

The present invention provides methods for assessing the anti-inflammatory efficacy of drug substances using one or more biomarkers of inflammation. Kits for performing the disclosed methods are also provided.

### CROSS-REFERENCE TO RELATED PATENTS AND PATENT APPLICATIONS

The present application is a non-provisional application of and claims the benefit of U.S. Provisional Application No. 60/808,396, filed 25 May 2006, which is incorporated herein by reference in its entirety.

### FIELD OF THE INVENTION

The present invention relates generally to biomarkers of inflammation, and more particularly to the use of biomarkers of inflammation as indicators of drug efficacy.

### BACKGROUND OF THE INVENTION

The correlation of a new chemical entity's efficacy with clinical endpoints makes human trials a long and expensive process. Furthermore, the results of human trials often depend upon subjective testimony from the trial candidates themselves, with little or no biochemical or physiological data to substantiate claims. Significant efforts are ongoing towards identification of biomarkers that can serve as objective surrogate end points to indicate the efficacy of a drug.

Inflammation has been accepted to be a central theme of many diseases including cardiovascular diseases, arthritis, metabolic disorders and cancer. As such, many pharmaceutical companies attempt to incorporate anti-inflammatory effects into their drug candidates.

Because of the importance of inflammation in human disease, it would be desirable to provide methods for assessing the efficacy of drug substances using biomarkers of inflammation.

### SUMMARY OF THE INVENTION

The present invention provides methods for assessing the efficacy of a drug substance using one or more biomarkers of inflammation. Such methods allow an early proof-of-concept on the anti-inflammatory effects of new chemical entities, as well as allowing marketed drugs to be assessed for anti-inflammatory activity. The methods can also be used to for dose selection in clinical trials and dose titration of marketed drugs.

Accordingly, one aspect of the present invention is directed to a method for assessing the efficacy of a drug substance in a subject, comprising the steps of:

collecting a first biological sample from a subject prior to treatment with a drug substance;

exposing said first biological sample to an inflammatory agent, thereby inducing expression of an inflammation biomarker in said first sample;

measuring the level of said inflammation biomarker in said first biological sample;

administering said drug substance to said subject;

collecting a second biological sample from said subject following administration of said drug substance;

exposing said second biological sample to said inflammatory agent; and

measuring the level of said inflammation biomarker in said second biological sample,

wherein a decrease in the level of said inflammation biomarker in said second biological sample as compared to said first biological marker indicates that the drug substance is efficacious.

The present invention is also directed to further downstream uses of the methods herein, as well as kits for carrying out the methods.

### BRIEF DESCRIPTION OF THE DRAWINGS

FIG. 1 is a bar graph demonstrating the anti-inflammatory effect of a drug substance on the LPS-induced level of TNF in whole blood from health subjects enrolled in a Phase I clinical trial.

### DETAILED DESCRIPTION OF THE INVENTION

The present invention provides methods for assessing the efficacy of a drug substance using one or more biomarkers of inflammation. Applicants have found that the anti-inflammatory effect of these substances, and hence their potential efficacy in humans, may be assessed by measuring levels of inflammation biomarkers prior to and following drug administration.

Accordingly, one aspect of the present invention is directed to a method for assessing the efficacy of a drug substance in a subject, comprising the steps of:

collecting a first biological sample from a subject prior to treatment with a drug substance;

exposing said first biological sample to an inflammatory agent, thereby inducing expression of an inflammation biomarker in said first sample;

measuring the level of said inflammation biomarker in said first biological sample;

administering said drug substance to said subject;

collecting a second biological sample from said subject following administration of said drug substance;

exposing said second biological sample to said inflammatory agent; and

measuring the level of said inflammation biomarker in said second biological sample,

wherein a decrease in the level of said inflammation biomarker in said second biological sample as compared to said first biological marker indicates that the drug substance is efficacious.

Subjects

The term “subject” as used herein refers to any animal for whom diagnosis, treatment, or therapy is desired. The term “animals” as used herein refers to humans and other mammals, as well as other animals. Human subjects may be those enrolled in clinical trials (e.g., Phase I, II, III and/or IV).

Biological Sample

As used herein, “biological sample” includes urine, whole blood (EDTA or heparin treated), plasma, serum, saliva, tissue biopsies, cerebrospinal fluid, peripheral blood mononuclear cells (PBMCs), monocytes and mixtures thereof. PBMCs may be harvested from EDTA- or heparin-treated, non-coagulated venous blood using methods known to those skilled in the art, such as Ficoll-Hypaque density centrifugation.

Inflammatory Agents

To induce expression of an inflammatory biomarker, biological samples from a subject prior to and following drug administration (at single or multiple time points) are exposed to an inflammatory agent. Any inflammatory agent can be used, so long as it induces expression of an inflammatory biomarker capable of being measured. A preferred method of inducing expression of an inflammation biomarker in the biological sample is by exposing the sample to endotoxin, such as lipopolysaccharide (LPS).

Inflammation Biomarker

Any inflammation biomarker capable of being measured can be used in the methods of the present invention.

Suitable inflammation biomarkers include inflammatory proteins, such as cytokines, including TNF, IL-2, IL-6, IL-8, VCAM, ICAM, CRP, MCP-1 and combinations thereof.

Preferred inflammation biomarkers include TNF, IL-8 and ICAM.

Measuring Means

Means for measuring levels of inflammation biomarkers in a biological sample comprise methods well known in the art. The amount of inflammation biomarker can be measured at the protein level or the mRNA level. Non-limiting protein methods include enzyme-linked immunosorbent assays (ELISAs), radioimmunoassays (RIAs), Western blots, enzymatic assays and chromatograph-based separation systems. Non-limiting mRNA methods include RT-PCR, Northern blots, microchip gene arrays and ribonuclease protection assays. The amount of inflammation biomarker is preferably measured at the mRNA level. A particularly preferred method is real-time RT-PCR using SYBR Green or TaqMan® probes.

Analysis

The levels of inflammation biomarkers are analyzed using standard statistical tools. LPS-induced levels can be compared in multiple manners, e.g., −LPS vs +LPS samples provide the effect of LPS on the particular biological samples; pre-drug administration vs post-drug administration in the same subject provides the anti-inflammatory effect of the drug; comparison of the profiles among different subjects in a study population provides the differential effect of the drug vs placebo. In fact, once the data is generated, analysis can be done in any number ways to obtain critical efficacy and safety information (e.g., inflammatory response vs drug response, drug response vs age, drug response v. side effects, drug response for AUC, drug response over time, etc.).

Applications

The methods described herein are capable of obtaining a general proof-of-concept on the anti-inflammatory effects of new chemical entities targeted for various therapeutic disorders. This can be useful in promoting the right molecules and discontinuing the ones which do not show any anti-inflammatory effects. In addition, the methods of the present invention can also used to assess the anti-inflammatory effects of marketed drugs that have already undergone gone safety and efficacy studies, thereby expanding the therapeutic horizon for which they are being utilized.

The methods of the present invention can also be used for dose selection based on the anti-inflammatory effect shown by the drug substance vs the ratio of required features. For example, the data from the multiple ascending dose (MAD) study of a Phase I clinical trial can be used to select the dosing regimen for Phase II. Dose titration can also be performed on an individual subject basis by monitoring the changes in inflammation biomarker levels over time.

Kits

A wide variety of kits may be prepared for performing the methods of the present invention. For example, a kit may include an inflammatory agent and means for measuring the level of one or more inflammation biomarkers in a biological sample.

When the measuring means comprises RT-PCR, the kit will preferably comprise amplification primers specific for the inflammation biomarker(s) of interest. Such kits may also comprise control amplification primers, RNA extraction reagents, labeled probes and various RT-PCR reagents (e.g., polymerases, nucleotides, buffers, etc.).

The kits may further comprise means for obtaining biological samples from a subject. Such means may comprise venous blood collection tubes and devices for collecting blood, sample tubes for urine or saliva, means for separating PBMCs from venous blood, and combinations thereof.

The kits may also comprise appropriate instructional materials for use. While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media may include addresses to internet sites that provide such instructional materials.

Specific embodiments according to the methods of the present invention will now be described in the following examples. The examples are illustrative only, and are not intended to limit the remainder of the disclosure in any way.

### EXAMPLES

### Example 1

A real-time LPS induction assay has been developed to assess the inflammatory effects of endotoxin on inflammation biomarkers in whole blood.

Whole blood is drawn from individuals after giving informed consent and stored in EDTA-coated or heparin-coated vacutainers. The blood is aliquoted and incubated with 0.05-10 ng LPS at 37° C. for 1-6 hr. A control aliquot is incubated with RPMI in place of LPS (−LPS). Incubation for 1 hr has shown maximal induction of inflammation. Following incubation, the blood samples are transferred to pre-labeled pax gene tubes and frozen at −80° C. to arrest transcription.

The frozen blood samples are allowed to thaw at room temperature and incubated for 14 hours at room temperature for lysis. The samples are mixed by inversion 5 times and the RNA is isolated following manufacturers protocol (Qiagen) followed by rigorous DNAse treatment using DNA-free (Ambion). The quality of RNA is routinely assessed by electrophoresis in formaldehyde-based agarose gel electrophoresis and spectrophotometer readings in 1M Tris. An intact 28 and 18S RNA bands in 2:1 ratio as indicated in the gel and a ratio of 2.0 in 1M Tris is used as criteria for RNA QC. 2-3 samples are randomly picked for assessment by electrophoretic evaluation. All the samples from each set can be processed together to avoid technical variability.

SYBR green based quantitative real time PCR is used to assess the transcript levels of inflammatory genes as biomarkers of inflammation. Primers specific for inflammatory genes (and a control housekeeping gene, such as ACTB) are designed using the Primer 3 software following standard criteria (e.g., length, Tm, GC content, etc.). Two-hundred ng of RNA from each sample is reverse transcribed using Roche Transcriptor reverse transcriptase. The quality and quantity of cDNA is assessed by spectrophotometer readings. Two-hundred ng of cDNA is used for SYBR green based quantitative PCR assay. The assays are performed in duplicate and the average threshold cycle (CT) of the duplicates is used as sample CT in data analysis by ddCT method of relative quantification. SD of the CTs from the duplicates of each sample is used as a QC for the experimental variability. Briefly, dCT is calculated by subtracting the CT obtained for the housekeeping gene (called the normalizer) from the CT for the inflammation biomarker. ddCT is then calculated by subtracting the dCT of the control samples (called the calibrator) from the dCT of other samples. To assess the effect of LPS, −LPS serves as calibrator. In the case of drug effect, the pre-drug profile serves as calibrator for the post drug expression profile. In case of same time point study, the placebo samples serve as calibrators. Expression levels for each sample are calculated as 2 and represented as % expression levels relative to calibrators (minus LPS, placebo or pre-drug).

Irrespective of LPS amount or time of incubation, TNF and IL-6 showed good fold-induction in a dose-dependent fashion. However, with IL-6, some subjects showed very low basal levels of IL-6, making level assessment difficult. To identify other genes to serve as secondary inflammation biomarkers to TNF, CRP, MCP-1, IL-8, IL-2 and ICAM were assessed in the LPS induction assay. Of these, both IL-8 and ICAM showed dose-dependent induction by LPS. The LPS induction assay has been shown to be reproducible by other personnel in other laboratory environments.

### Example 2

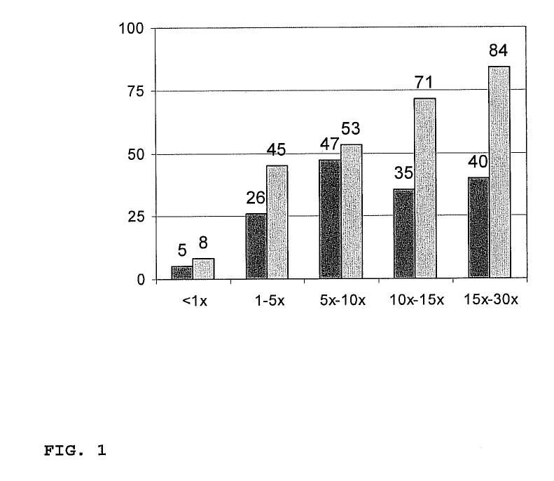

The LPS induction assay described above was performed on healthy subjects in a Phase I clinical trial. The clinical trial consisted of 8 subjects in every cohort, 6 on actives and 2 on placebos. Subjects in two cohorts (25 mg and 250 mg cohort) were subjected to the LPS induction assay, at two time points, pre-dosing and post-dosing (cohort 1, 25 mg, 6 hrs post-doing; cohort 3, 250 mg, 24 hrs post-dosing). The data was compared as pre- and post-dosing and plotted in response to induction levels. As depicted in FIG. 1, the x axis is fold TNF induction and the y axis is % inhibition of TNF expression levels (left bars are cohort 1, right bars are cohort 3). The anti-inflammatory effect of the drug substance on the LPS-induced levels of TNF is clearly seen in relation to the level of biomarker induction and the drug dose. The study clearly demonstrates that the methods described herein can be used to assess the anti-inflammatory activity of a tested drug substance in an MAD study, thereby providing an early proof-of-concept for the drug.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A method for assessing the efficacy of a drug substance in a subject, comprising the steps of:
collecting a first biological sample from a subject prior to treatment with a drug substance;
exposing said first biological sample to an inflammatory agent, thereby inducing expression of an inflammation biomarker in said first sample;
measuring the level of said inflammation biomarker in said first biological sample;
administering said drug substance to said subject;
collecting a second biological sample from said subject following administration of said drug substance;
exposing said second biological sample to said inflammatory agent; and
measuring the level of said inflammation biomarker in said second biological sample,
wherein a decrease in the level of said inflammation biomarker in said second biological sample as compared to said first biological marker indicates that the drug substance is efficacious.

2. The method of claim 1, wherein the subject is enrolled in a clinical trial.

3. The method of claim 2, wherein the subject is enrolled in a Phase I clinical trial.

4. The method of claim 1, wherein the inflammatory agent is endotoxin.

5. The method of claim 4, wherein the inflammatory agent is LPS.

6. The method of claim 1, wherein the inflammation biomarker is a cytokine.

7. The method of claim 6, wherein the cytokine is TNF or IL-8.

8. The method of claim 1, wherein the steps of measuring the levels of inflammation biomarker is done at the level of mRNA.

9. The method of claim 8, wherein the steps of measuring the levels of inflammation biomarker is performed using real-time RT-PCR.

10. The method of claim 9, comprising the use of using SYBR Green or TaqMan® probes.

11. The method of claim 1, wherein the first and second biological samples comprise whole blood.

12. The method of claim 1, wherein the drug substance is a new chemical entity.

13. The method of claim 1, wherein the drug substance is a marketed drug.

14. A kit for performing the method of claim 1, comprising an inflammatory agent and means for measuring the level of the inflammation biomarker.

15. The kit of claim 14, wherein the means for measuring the level of the inflammation biomarker comprise RT-PCR.

16. The kit of claim 15, comprising amplification primers specific for the inflammation biomarker.

17. The kit of claim 16, further comprising control amplification primers, RNA extraction reagents, labeled probes, polymerases, nucleotides, buffers and combinations thereof.

18. The method of claim 2, wherein a dosing regimen for the drug substance is selected in a subsequent clinical trial.

19. The method of claim 1, wherein the dose of the drug substance is titrated in an individual subject.

20. The method of claim 1, further comprising assessing one or more of the following:
a) an effect of the inflammatory agent on the biological sample;
b) an anti-inflammatory effect of the drug substance in an individual subject;
c) an anti-inflammatory effect of the drug substance compared to placebo in a population of subjects;
d) an anti-inflammatory effect of the drug substance compared to the extent of the induced inflammatory response;
e) an anti-inflammatory effect of the drug substance compared to age in a population of subjects;
f) an anti-inflammatory effect of the drug substance compared to its side effects;
g) an anti-inflammatory effect of the drug substance in an individual subject over time; and
h) an anti-inflammatory effect of the drug substance compared to its AUC.
